(19) United States
(12) Patent Application Publication
(10) Pub. No.: US 2016/0299130 A1
(43) Pub. Date: Oct. 13, 2016

(54) Telocinobufagin (TCINO) in the Diagnosis and Pathogenesis of Preeclampsia, Traumatic Brain Injury and Acute Respiratory Distress Syndrome

(71) Applicant: Jules B. PUSCHETT, Houston, TX (US)

(72) Inventor: Jules B. PUSCHETT, Houston, TX (US)

(21) Appl. No.: 15/092,715

(22) Filed: Apr. 7, 2016

Related U.S. Application Data
(60) Provisional application No. 62/145,475, filed on Apr. 9, 2015.

(51) Int. Cl. G01N 33/53

(52) U.S. Cl. CPC G01N 33/5308; G01N 2800/368; G01N 2800/125; G01N 2800/28; G01N 2400/00

(57) ABSTRACT

Certain embodiments are directed to methods of identifying patients with PE, TBI, or ARDS by detecting elevated levels of MBG, TCINO, and CINO and treating the same with anti-MBG agents.

### CROSS REFERENCE TO RELATED APPLICATIONS

This application claims the benefit of U.S. Provisional Patent Application No. 62/145,475, filed Apr. 9, 2015, which is herein incorporated by reference in its entirety.

### SUMMARY

Bufodienolides are involved in the pathogenesis of preeclampsia (PE) (Vu et al., Am J Nephrol 25:520-528, 2005; Puschett et al., Biochim Biophys Acta (1802):1246-53, 2010; Uddin et al., Biochim Biophys Acta 1812(1):49-58, 2011; Agunanne et al., Am J Perinatol 28(7):509-14, 2011), traumatic brain injury (TBI) (Shapiro et al., The Annual Meeting of the American Society for Clinical Pharmacology and Therapeutics, Dallas, Tex., Mar. 2-5, 2011. ASCPT Annual Meeting Program Booklet, p. '78) and the acute respiratory distress syndrome (ARDS) (Fargo et al., The Annual Meeting of the Southern Society for Clinical Investigation, New Orleans, La., Feb. 20-22, 2014. J Invest Med 62:544-45, 2014). Bufodienolide, marinobufogenin (MBG) (Ianosi-Irimie et al., Clin Exp Hypertens 8:605-17, 2005; Vu, et al., Exp Biol Med 231:215-20, 2006) and cinobufatalin (CINO) (Puschett et al., Am J Perinatology) are involved in disturbances in which volume expansion (VE) plays a role such as in PE (Puschett et al., Medical Hypotheses 67:1125-32, 2006; Danchuk et al., Am J Nephrol 28:8-13, 2008). Volume expansion (VE) is a major component of the complications of chronic kidney (renal) failure (CKD), since, as the kidney disease progresses, fluid excretion declines, leading to VE. Thus, because patients, especially those that have reached en-stage renal disease (ESRD) become volume overloaded, the levels of the bufodienolides rise in their blood (Gonick et al., Clin Exp Hypertens 20:617-27, 1998). Such is the case for telocinobufagin (TCINO), which has been reported to reach levels that are seven times those of MBG (Komiyama et al. Clin Biochem, 2005, 38:36-45).

A panel of tests would include all 3 of the bufodienolides, MBG, CINO, and TCINO (FIG. 2). All 3 of these bufodienolides have been isolated from the ancient Chinese medicine Chan Su (Li et al., J Sop Sci 33:1325-30, 2010; Liang et al., Pharmaceutical Biomedical Analysis 46:442-48, 2008). Provided in FIG. 2 are examples of antagonists for each of these 3 bufodienolides. They share the property that, in each case, the chemical structures of the agonists have been altered to produce the specific antagonist of each. Thus, resibufogenin (RBG) has been determined to be the antagonist of MBG; cinobufagin (CBG) is an antagonist of cinobufatalin; and bufalin (BUF) is an antagonist of TCINO (FIG. 2). In each case, the antagonist of MBG, CINO and TCINO consists in the absence of the hydroxyl group at the Beta-5 position of the respective agonist(s).

Certain embodiments are directed to a panel of tests including TCINO, MBG, and CINO for the diagnosis of PE, TBI, and ARDS. The expanded panel increases the efficiency of diagnosis of PE, TBI, and ARDS while decreasing the rate of false diagnosis when using MBG or CINO alone. The appropriate bufodienolide antagonist can be administered to counteract the effects of elevated bufodienolide(s). In certain aspects the levels of bufodienolides can be used to determine which bufodienolide antagonist to administer.

Embodiments are directed to a diagnostic kit for assessing the levels of MBG, CINO, and TCINO in urine, blood, or urine and blood samples. The kit comprises bufodienolide binding agents, e.g., MBG, CINO, and TCINO specific antibodies. Such a diagnostic panel can be used to assess bufodienolide levels associated with PE, TBI, and/or ARDS. Accordingly, if one or more of their levels is elevated, then appropriate antagonists can be selected for therapy.

As used herein, the term “analyte” generally refers to a substance to be detected. For instance, analytes may include substances such as small molecules or proteins. Analytes include, but are not limited to, organic compounds, proteins, and peptides. Specific examples of analytes include MBG, CINO, and TCINO.

As used herein, the term “test sample” or “sample” generally refers to a biological material suspected of containing an analyte of interest. The test sample may be derived from a biological source, such as a biological fluid, including, blood or urine. The test sample may be used directly as obtained from the biological source or following a pretreatment to modify the character of the sample. For example, such pretreatment may include preparing plasma from blood and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustrates the cardiac glycosides (or cardiotonic steroids). The cardenolides are on the left and the bufodienolides on the right.

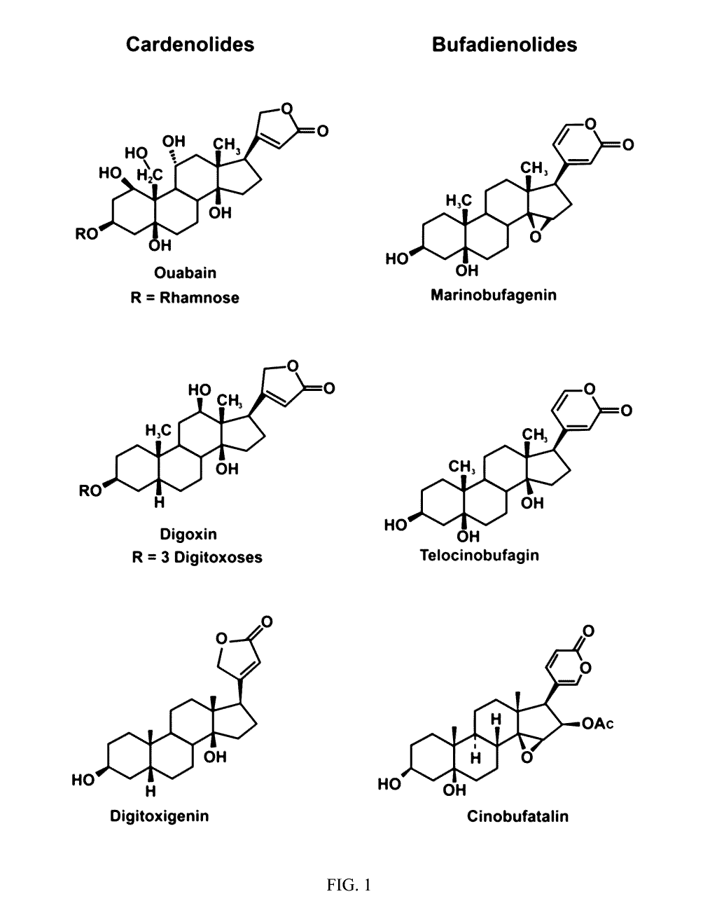

FIG. 2. Illustrates the three agonist bufodienolides of interest, which are shown in the top structure of each group. Their antagonists are pictured in the bottom of each panel.

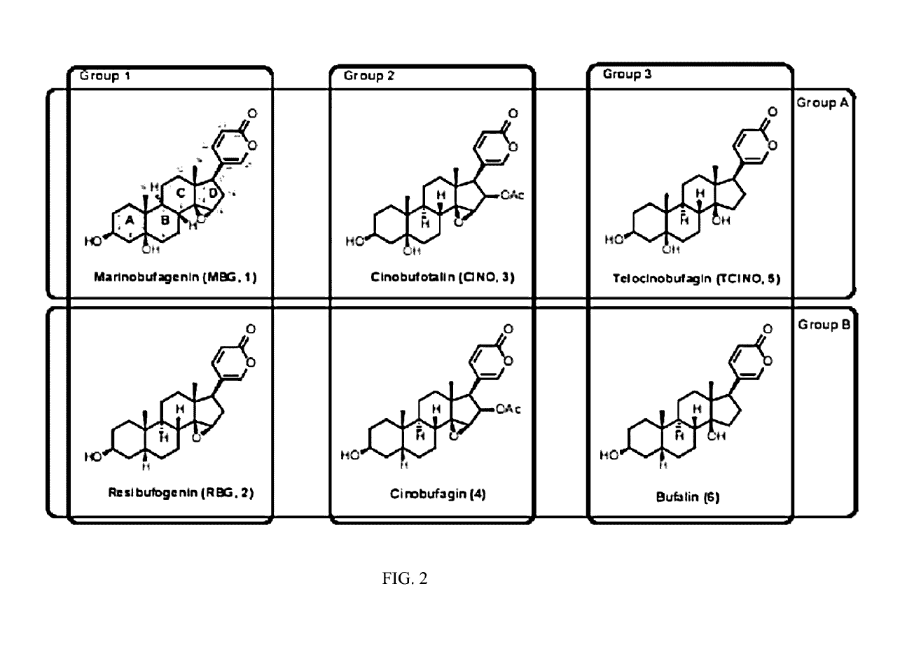

### DESCRIPTION

Bufadienolides were discovered in amphibians and extracted from plants. They are thought to act by virtue of their ability to inhibit Na+/K+-ATPase activity (Flier et al., Science 1980, 208:503-05). Several bufadienolides have been suggested as candidate sodium pump ligands (SPLs) in mammals, including marinobufagenin (MBG), which acts in vitro as a vasoconstrictor (Fedorova et al., Am. J. Hypertens. 1997, 10:929-35; Lopatin et al., J. Hypertens. 1999, 17:1179-87). Enhanced MBG production occurs in pathological states associated with fluid retention, including essential and salt-sensitive hypertension, preeclampsia, and uremic cardiomyopathy (Gonick et al, Clin. Exp. Hypertens. 1998, 20: 617-27, Bagrov et al., Hypertension 1998, 31:1097-1103; Fedorova et al., Hypertension 2001, 37:462-66; Lopatin et al., J. Hypertens. 1999, 17:1179-87; Fedorova et al, Circulation 2002; 105: 1122-27; Kennedy et al., Hypertension 2006, 47:448-495).

Bufodienolides (e.g., Formulas I, II, and III below) and related substances called the cardenolides (e.g., Formulas IV, V, and VI below) are termed the “cardiotonic steroids” or “cardiac glycosides.” They are similar in general structure but different in specific structure and function. However, all of the cardiotonic steroids have the ability to inhibit the ubiquitous enzyme sodium/potassium ATPase (Na+/K+ ATPase).

Certain aspects described herein relate in part to marinobufagenin (MBG), the most extensively studied of the bufodienolides. MBG is elevated in disturbances that result from excessive volume expansion (Gonick et al. Clin. Exp. Hypertens. 20: 617-627, 1998; Ianosi-Irimie et al. Clin. Exp. Hypertens. 8: 605-617, 2005; Vu et al. Exp. Biol. Med. 231: 215-220, 2006). An immunoassay has been developed to measure MBG in both serum and urine (Abi-Ghanem et al. Journal of Immunoassay and Immunochemistry 32: 31-46, 2011). Certain embodiments are directed to the measurement of MBG for determining whether or not excessive volume expansion exists.

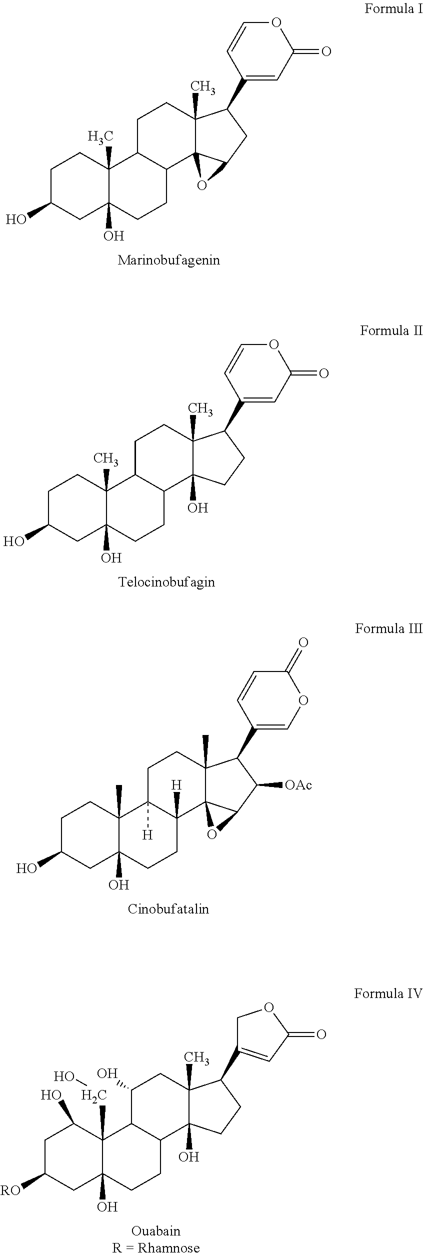

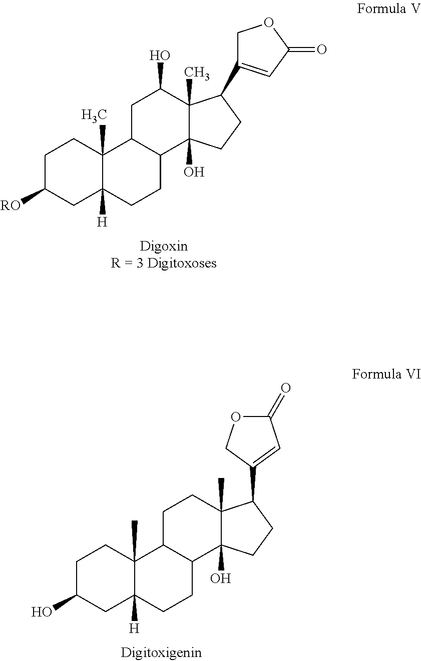

In certain aspects the concentration or levels of MBG, CINO, and TCINO can be determined and the pathogenesis of PE, TBI, or ARDS determined in a patient based on these levels. Accordingly, appropriate therapy is prescribed based on the resulting levels of MBG, CINO, and TCINO.

MBG is elevated in preeclampsia and traumatic brain injury. It has been shown that the administration of the antagonist of MBG, resibufagenin (RBG) is therapeutically effective. RBG administration results in an amelioration of tissue injury in a rodent brain contusion model when given 1 hour after the insult and prevents preeclampsia in animal models. RBG antagonizes MBG activity and may be an effective treatment for hypertension, ARDS, traumatic brain injury, and preeclampsia.

In certain aspects if a patient is tested and is positive for elevated levels of one or more of MBG CINO, and TCINO, then the patient is administered an antagonist of the elevated bufodienolide(s) as a PE, TBI, or ARDS therapy.

In certain aspects, elevated levels of MBG CINO, and TCINO are at least 30, 40, 50, 60, 70, 80, 90, 100, 200% or more than normal levels or a reference level. In other aspects elevated MBG, CINO, and TCINO levels are at least 1.5, 2, 3, 4, 5, 6, 7, 8, 9, 10 or more times the normal levels or a reference level. In certain embodiments MBG, CINO, and TCINO are detected or measured in blood or urine.

MBG, CINO, and TCINO can be detected using a variety of assays including, but not limited to immuno-detection, microchip, or lateral flow based methods. Immuno-based assays include, but are not limited to radiolabeled, enzyme, fluorescence, dot blot, chemiluminescence, dip-stick, or biosensor assays. See, for example, Principles and Practice of Immunoassays, Price, C. P. and Newman, D. J. (Eds.), Stockton Press, 1997; The Immunoassay Handbook; 2nd Edition, Wild, D. (Ed.), Nature Publishing Group, London, 2001.

Methods for measuring MBG, CINO, and TCINO in the blood or urine can provide sensitivities in the pg/ml (picograms per milliliter) range or pg/mg creatinine, respectively. In certain aspects the MBG, CINO, or TCINO detection methods employ an MBG, CINO, or TCINO specific ELISA assay. In certain aspects, antibodies are used to detect the presence of MBG, CINO, or TCINO in an original or processed sample obtained from a subject. Samples obtained from a subject may include, for example, cells, tissue, blood, serum, or urine. For example, a sample can be blood or urine collected from a subject. A sample can be analyzed directly or extracted before analysis.

In certain aspects a sample is contacted with an effective amount of one or more antibody/antibodies and the sample is screened to detect an antibody-MBG or antibody-CINO or antibody-TCINO complex, such as detecting the binding reaction between the antibody and the MBG or CINO or TCINO. Detection of an antibody-antigen complex or binding reaction indicates that the sample contains MBG, CINO, or TCINO. In some embodiments, the one or more antibody is labeled with a detectable moiety, such as a fluorescent label, so that its signal changes upon binding to MBG, CINO, or TCINO.

In other embodiments, MBG, CINO, or TCINO in the sample is immobilized on a surface and detected. In certain aspects MBG, CINO, or TCINO is immobilized prior to introduction of the labeled antibody, and the amount of the signal, corresponding to the amount of bound labeled antibody, correlates to the amount of MBG, CINO, or TCINO in the sample. In still other embodiments, MBG, CINO, or TCINO is captured by an immobilized unlabeled first antibody, after which a labeled second antibody is introduced to bind to the captured MBG, CINO, or TCINO and produce a signal in proportion to the amount of captured MBG, CINO, or TCINO. In other embodiments the binding of MBG, CINO, or TCINO is detected by altering the properties of the surface to which it is directly or indirectly bound.

In general, an antibody can be used as a labeled primary reagent in a direct assay or as an unlabeled reagent to be detected by a secondary developing antibody conjugate, such as labeled anti-rabbit antibody, in an indirect assay (Abi-Ghanem et al., Journal of Immunoassay and Immunochemistry, 32:31-46, 2011). Additionally, an antibody can be used in a competition assay to detect MBG, CINO, or TCINO in a sample. For example, MBG, CINO, or TCINO in a sample extract is captured by an unlabeled antibody immobilized on the surface of an ELISA well and then detected by a labeled antibody of the same or different kind and/or specificity. Alternatively, the sample can be suspended in a buffer and mixed directly with an antibody, thus allowing the antibody to form an immune complex with MBG, CINO, or TCINO. The reduction of free antibody due to complex formation can then be determined in a second step, based on solid-phase ELISA with purified MBG, CINO, or TCINO, by comparing the relative reactivity of free residual antibody left over after sample incubation (sample reactivity) to that of the same antibody when not mixed with the sample (reference reactivity). The ratio of sample to reference antibody reactivity will be inversely proportional to the amount of MBG, CINO, or TCINO in the sample.

In certain aspects, methods of the invention can be adapted for lateral flow assays and devices supporting such. Lateral flow assays, also known as immunochromatographic assays, are typically carried out using a simple device intended to detect the presence (or absence) of a target analyte in the sample. Most commonly these tests are used for medical diagnostics either for home testing, point of care testing, or laboratory use. Often produced in a dipstick format, these assays are a form of immunoassay in which the test sample flows along a solid substrate via capillary action. After the sample is applied to the test it encounters a colored or labeling reagent which mixes with the sample and transits the substrate encountering lines or zones which have been pretreated with an antibody or antigen or affinity reagent. Depending upon the analyte present in the sample the colored or labeling reagent can become bound at the test line or zone. Lateral flow assays can operate as either competitive or sandwich assays.

In still a further aspect, methods of the invention can be adapted for protein array assays and devices supporting such. Protein arrays or microarrays (also known as a biochip, or a protein chip) are measurement devices used in biomedical applications to determine the presence and/or amount of an analyte(s) in biological samples, e.g. blood, urine, swabs, tissues scrappings, etc. Typically, a number of different capture agents, most frequently monoclonal antibodies, can be deposited on a chip surface (glass or silicon) in an array. This format is also referred to as a microarray (a more general term for chip based biological measurement devices).

In yet another aspect the assay can be a microfluidic chip-based assay. Microfluidics is an important innovation in biochip technology. Since microfluidic chips can be combined with immunodetection and mass spectrometric analysis (See Wang et al., Lab Chip 13:4190-97, 2013; Baker et al. Bioanalysis 1(5): 967-75 2009; Wang et al., Anal. Chem. 72:832-839, 2000).

In certain embodiments, the invention also provides compositions comprising an anti-MBG agent or MBG antagonist; anti-CINO or CINO antagonist; or anti-TCINO or TCINO antagonist with one or more of the following: a pharmaceutically acceptable diluent, a carrier, a solubilizer, an emulsifier, and/or a preservative. Such compositions may contain an effective amount of at least one anti-MBG, anti-CINO, or anti-TCINO agent. The use of an anti-MBG agent in combination with other PE, TBI, or ARDS therapy is also included.

Acceptable formulation components for pharmaceutical preparations are nontoxic to recipients at the dosages and concentrations employed. In addition to the anti-hypertensive agents that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophobic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, 2,4-hexadienoic acid, or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The pharmaceutical composition to be used for in vivo administration is typically sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The above compositions can be administered using conventional modes of delivery including, but not limited to, intravenous, intraperitoneal, oral, intraarterial, and intrapleural. In certain aspects an anti-hypertensive agent will be administered orally. When administering the compositions by injection, the administration may be by continuous infusion or by single or multiple boluses. For parenteral administration, the anti-hypertensive agents may be administered in a pyrogen-free, parenterally acceptable solution comprising the desired anti-hypertensive agents in a pharmaceutically acceptable vehicle. A particularly suitable vehicle for parenteral injection is one in which one or more anti-MBG and/or anti-angiotensin agents are formulated as a sterile solution, properly preserved.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers that are conventionally employed in pharmaceutical compositions, such as DMSO, oil, sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts. In certain aspects the composition utilize 10 to 30% DMSO and/or oil (e.g., sesame oil).

The components used to formulate the pharmaceutical compositions are preferably of high purity and are substantially free of potentially harmful contaminants (e.g., at least National Food (NF) grade, generally at least analytical grade, and more typically at least pharmaceutical grade). Compositions for parental administration are also sterile, substantially isotonic and made under GMP conditions.

For the compounds of the present invention, alone or as part of a pharmaceutical composition, such doses are between about 0.001 mg/kg and 1 mg/kg body weight, preferably between about 1 and 100 μg/kg body weight, most preferably between 1 and 10 μg/kg body weight.

Therapeutically effective doses will be easily determined by one of skill in the art and will depend on the severity and course of the disease, the patient's health and response to treatment, the patient's age, weight, height, sex, previous medical history and the judgment of the treating physician.

1. A method for assessing patients for PE, TBI, or ARDS comprising (a) contacting a patient sample with a first binding reagent that specifically binds marinobufagenin (MBG) and a second binding reagent that specifically binds TCINO; (b) detecting and quantitating MBG-binding reagent complexes and TCINO-binding reagent complexes; and (c) classifying a patient as having PE, TBI, or ARDS if MBG, TCINO, or MBG and TCINO levels are elevated.

2. The method of claim 1, further comprising contacting a patient sample with a third binding reagent that specifically binds CINO.

3. The method of claim 2, wherein the MBG, TCINO, and CINO binding reagents are coupled to a substrate.

4. The method of claim 2, further comprising administering a PE, TBI, or ARDS treatment to a subject having elevated levels of MBG, TCINO, CINO, MBG and TCINO, MBG and CINO, TCINO and CINO, or MBG, TCINO, and CINO.

5. The method of claim 2, wherein the MBG, TCINO, and CINO level is measured using an ELISA assay.

6. The method of claim 2, wherein the MBG, TCINO, and CINO level is measured using a lateral flow assay.

7. The method of claim 2, wherein an elevated blood level of MBG, TCINO, and CINO is greater than 45 pg/ml or elevated urine level is greater than 450 pg MBG/mg creatinine.

8. A kit comprising reagents for the detection of MBG, TCINO, and CINO.
